(19) United States
(12) Patent Application Publication
(10) Pub. No.: US 2018/0360898 A1
(43) Pub. Date: Dec. 20, 2018

(54) USE OF CRANBERRY DERIVED PHENOLIC COMPOUNDS AS ANTIBIOTIC SYNERGIZING AGENT AGAINST PATHOGENIC BACTERIA

(71) Applicant: THE ROYAL INSTITUTION FOR THE ADVANCEMENT OF LEARNING/MCGILL UNIVERSITY, Montréal, CA (US)

(72) Inventors: Nathalie TUFENKJI, Laval (CA); Vimal MAISURIA, Montreal (CA)

(21) Appl. No.: 16/060,683

(22) PCT Filed: Dec. 9, 2016
(86) PCT No.: PCT/CA2016/051447

§ 371 Date: Jun. 8, 2018

Related U.S. Application Data
(60) Provisional application No. 62/366,666, filed on Jul. 26, 2016.
(60) Provisional application No. 62/266,334, filed on Dec. 11, 2015.

(51) Int. Cl. A61K 36/45; A61P 31/04

(52) U.S. Cl. CPC A61K 36/45; A61P 31/04

(57) ABSTRACT

This present disclosure relates to the use of cranberry derived proanthocyanidins as antibiotic synergizing agent to mitigate multidrug resistance and biofilm formation in different pathogenic bacteria. The synergistic combination of antibiotic and proanthocyanidins could treat bacterial infections using a lower dose of antibiotics to prevent biofilm formation and proliferation of microorganisms, with defined modes of action.

### CROSS-REFERENCE TO RELATED APPLICATIONS

This application is a U.S. National Phase of International Application No. PCT/CA2016/051447, filed on Dec. 9, 2016 and claiming priority from U.S. provisional patent applications 62/266,334 filed Dec. 11, 2015, and 62/366,666 filed Jul. 26, 2016 and this application claims priority to and the benefit of the above-identified applications, each of which are incorporated by reference herewith in their entirety.

### TECHNICAL FIELD

The present description relates to the use of a cranberry extract for treating a bacterial infection.

### BACKGROUND ART

In light of the global rise in antibiotic resistance of many pathogenic bacteria, the synergistic anti-microbial role of foods warrants further consideration. Bacteria have evolved multiple strategies for causing infections that include undertaking motility, producing virulence factors, adhering to surfaces, developing communities called biofilms, and bacterial persistence.

There is thus a need to be provided with new antibacterial composition.

### SUMMARY

In accordance with the present disclosure, there is now provided a composition comprising a cranberry extract and a carrier for treating a bacterial infection.

In an embodiment, the composition described herein further comprises an antibiotic.

In another embodiment, the cranberry extract comprises proanthocyanidins, flavanols, anthocyanidins, procyanidins, terpenes, hydroxybenzoic acids, hydroxycinnamic acids, flavonoids, tannins, phenolic acids, other bioactive polyphenols or combinations thereof.

In an additional embodiment, the cranberry extract is from at least one of Vaccinium macrocarpon, Vaccinium oxycoccos, Vaccinium microcarpum, and Vaccinium erythrocarpum.

In a further embodiment, the cranberry extract is from Vaccinium macrocarpon.

In another embodiment, the antibiotic is an aminoglycoside, a polyketide, a macrolide, a fluoroquinolone or a β-lactam.

In an embodiment, the antibiotic is gentamicin, kanamycin, tetracycline, or azithromycin.

It is also provided the use of a cranberry extract for treating a bacterial infection.

In an embodiment, the antibiotic and the cranberry extract are formulated for an administration concurrently or separately.

It is further provided the use of the composition encompassed herein for decreasing multidrug resistance.

It is further provided the use of the composition encompassed herein for decreasing an antibiotic resistance.

It is further provided the use of the composition encompassed herein for decreasing biofilm formation.

It is also provided a method of treating a bacterial infection, comprising administering a cranberry extract to a subject.

In an embodiment, the subject is an animal or a human.

### BRIEF DESCRIPTION OF THE DRAWINGS

Reference will now be made to the accompanying drawings.

FIG. 1 illustrates that cranberry derived proanthocyanidins (cPACs) synergize with the antibiotic. Representative heat plots showing synergistic growth inhibition of (A) Escherichia coli CFT073 and (B) Pseudomonas aeruginosa PAO1 at different concentrations of cPAC fraction-1 and gentamicin are shown.

FIG. 2 illustrates the synergistic interaction of cranberry-derived materials with antibiotic for growth inhibition. MICs were determined for combination of (A) cPAC#1, (B) cPAC#2, (C) cPAC#3, or (D) cPAC#4, in combination with each antibiotic against E. coli CFT073 and P. aeruginosa PAO1. A FIC index of indicates synergy (values shown in blue), a FIC index of ≥0.5 and indicates no interaction/indifference, and a FIC index of >4 indicates antagonism. Gen: gentamicin; Tet: tetracycline; Azt: azithromycin; Kan: kanamycin; Cip: ciprofloxacin; Amp: ampicillin.

FIG. 3 illustrates growth curves for (A-D) E. coli CFT073 and (E-H) P. aeruginosa PAO1 with cPACs or gentamycin. Bacteria grown in the presence of (A, E) cPAC#1, (B, F) cPAC#2, (C, G) cPAC#3, (D, H) cPAC#4 or gentamicin. The graph shows the normalized OD600=OD600−initial OD600 versus time for bacteria grown in MHB-II broth (control) or with cPAC alone (concentration as indicated) or with gentamicin (MIC 2 μg/mL) alone. Data shown in growth curves are averages of n=3 with shaded S.D.

FIG. 4 illustrates the effect of each cPAC fraction with and without gentamicin on biofilm formation of E. coli CFT073. The graph presents normalized biofilm levels (OD570 nm/cell OD600 nm) versus different sub-inhibitory concentrations of gentamicin for E. coli CFT073 grown in MHB-II medium (control) or in MHB-II medium amended with sub-inhibitory concentrations of cPAC#1, cPAC#2, cPAC#3, or cPAC#4, with and without gentamicin. Error bars show the standard deviations from values obtained from three replications. Statistically significant differences are indicated for each sample treated with each cPAC fraction and gentamicin compared to the control (sample treated with the corresponding concentration of gentamicin only) (**, P<0.01; Two-way ANOVA) and also for samples treated with each cPAC fraction plus gentamicin compared to sample treated with the same concentration of each cPAC fraction without gentamicin (*, P<0.05; Two-way ANOVA).

FIG. 5 illustrates the effect of each cPAC fraction with and without gentamicin on biofilm formation of P. aeruginosa PAO1. The graph presents normalized biofilm levels (OD_{570 nm}/cell OD_{600 nm}) versus different sub-inhibitory concentrations of gentamicin for P. aeruginosa PAO1 grown in MHB-II medium (control) or in MHB-II medium amended with sub-inhibitory concentrations of cPAC#1, cPAC#2, cPAC#3, or cPAC#4, with and without gentamicin. Error bars show the standard deviations from values obtained from three replicates. Statistically significant differences are indicated for each sample treated with each cPAC fraction and gentamicin compared to the control (sample treated with the corresponding concentration of gentamicin only) (**, P<0.01; Two-way ANOVA) and also for samples treated with each cPAC fraction plus gentamicin compared to sample treated with the same concentration of each cPAC fraction without gentamicin (*, P<0.05; Two-way ANOVA).

FIG. 6 illustrates cPAC-mediated NPN uptake in (A) E. coli CFT073 and (B) P. aeruginosa PAO1. Bacterial cells were pretreated with cPAC#1, cPAC#2, cPAC#3, cPAC#4 or gentamicin (Gen) at sub-MICs. Enhanced uptake of NPN was measured by an increase in fluorescence (ex/em: 350 nm/420 nm) caused by partitioning of NPN into the hydrophobic interior of the outer membrane of pretreated bacterial cells. NPN is a hydrophobic fluorescent probe that fluoresces weakly in aqueous environment and strongly when it enters a hydrophobic environment such as the interior of a bacterial membrane. The background fluorescence of the medium was subtracted from all measurements, and the assay was repeated.

FIG. 7 illustrates the inhibition of multidrug efflux pump by cPACs in (A) E. coli CFT073 and (B) P. aeruginosa PAO1. Bacterial cells were pretreated without (control) and with 200 μg/mL cPAC#1, 200 μg/mL cPAC#2, 200 μg/mL cPAC#3, 200 μg/mL cPAC#4 or 100 μM CCCP (carbonyl cyanide m-chlorophenylhydrazone). EtBr efflux pump activity of the pretreated bacterial cells was monitored at room temperature for fluorescence intensity (ex/em: 530 nm/600 nm). Active efflux pump reduces accumulation of intracellular EtBr whereas inhibition of the efflux pump enhances accumulation of intracellular EtBr over time. The background fluorescence of the medium was subtracted from all measurements, and the assay was repeated independently in triplicate.

FIG. 8 illustrates the effect of each cPAC fraction on cell membrane integrity. Bacterial cells of E. coli CFT073 and P. aeruginosa PAO1 were pretreated separately with cPAC#1, cPAC#2, cPAC#3, cPAC#4 or cetyltrimethylammonium bromide (CTAB) at ½ MICs. The ratio of green to red fluorescence was normalized to that of the untreated control and expressed as a percentage of the control. The assay was repeated independently three times (*, P<0.05; t-test).

### DETAILED DESCRIPTION

It is provided a composition comprising a cranberry extract and a carrier for treating a bacterial infection.

Compounds derived from the American cranberry (V. macrocarpon L.) have been reported to exhibit anti-oxidant, anti-adhesion, anti-motility and anti-cancer activities. Herein, it is provided the anti-bacterial efficacy of the composition described herein comprising cranberry-derived proanthocyanidins and antibiotic and its potential in treating clinical and multiple drug resistant pathogenic bacterial strains.

Four different fractions of cranberry proanthocyanidins were tested, as provided by Ocean Spray Cranberries (see Table 1).

TABLE 1
Extent of antibiotic synergy of different cPAC samples against
Escherichia coli CFT073 and Pseudomonas aeruginosa PAO1

| | | % reduction in antibiotic usage | | | |
|---|---|---|---|---|---|
| cPAC | Bacterial | | Tetra- | Azith- | |
| samples* | Strains | Gentamicin | cycline | romycin | Kanamycin |
| cPAC-1 | CFT073 | 75% | 50% | 75% | 75% |
| | PAO1 | 75% | 75% | 50% | 75% |
| cPAC-2 | CFT073 | 88% | 75% | 50% | 94% |
| | PAO1 | 88% | 88% | 75% | 50% |
| cPAC-3 | CFT073 | 88% | 88% | 75% | 88% |
| | PAO1 | 75% | 0 | 75% | 0 |
| cPAC-4 | CFT073 | 88% | 88% | 75% | 94% |
| | PAO1 | 75% | 0 | 75% | 50% |

cPAC-1, ~95% (w/w) PACs enriched from cranberry fruit juice extract;
cPAC-2, ~95% (w/w) PACs enriched from cranberry extract;
cPAC-3, ~95% (w/w) PACs enriched from cranberry juice;
cPAC-4, 57% (w/w) PACs enriched polyphenolic extract containing flavonols and anthocyanins.

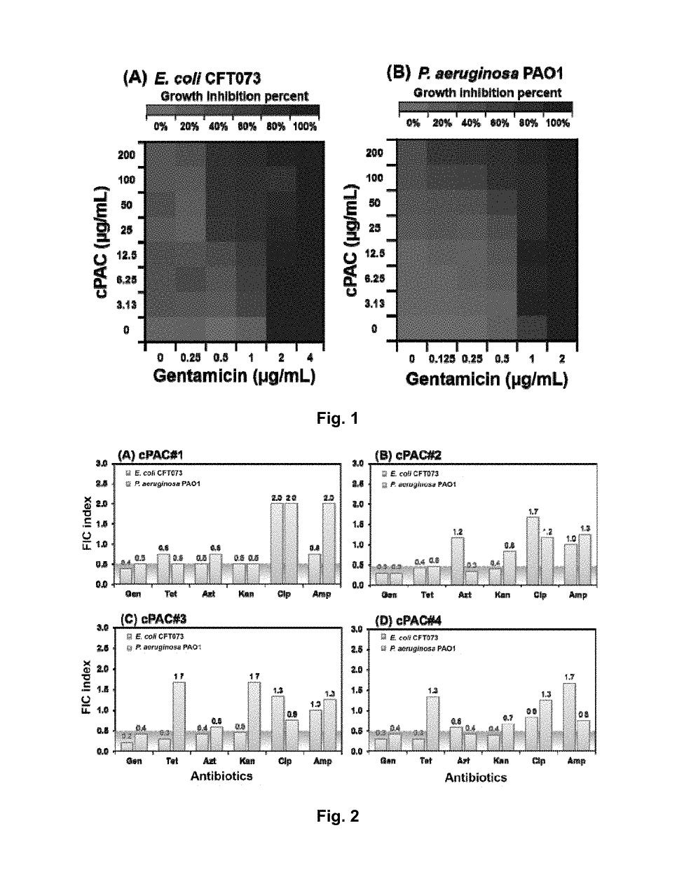

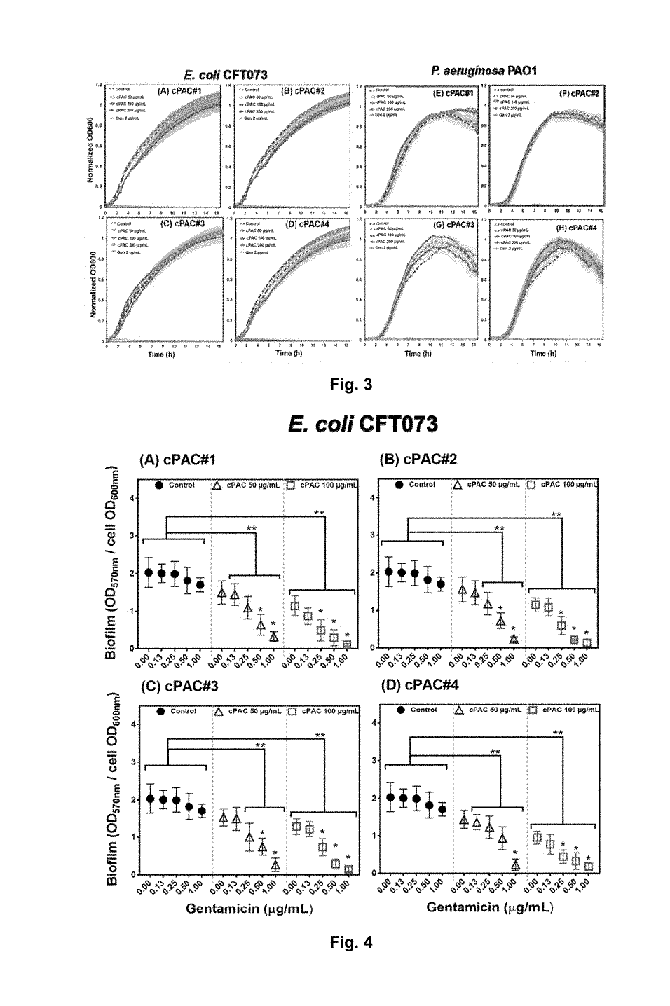

Experiments were conducted using combinations of proanthocyanidins and antibiotic (from different class of antibiotics such as aminoglycoside, polyketide, macrolide, fluoroquinolone and/3-lactam) to examine effects on growth inhibition of two different pathogenic bacteria (Escherichia coli CFT073 and Pseudomonas aeruginosa PAO1). The synergistic anti-bacterial properties of proanthocyanidins, which increase antibiotic susceptibility of each pathogenic bacterial strain at sub-inhibitory concentrations, is reported. Cranberry proanthocyanidins exhibit synergistic activity with two aminoglycoside antibiotics (gentamicin and kanamycin), a polyketide antibiotic (tetracycline), and a macrolide antibiotic (azithromycin) for growth inhibition of pathogenic bacteria (see Table 1, FIGS. 1 and 2). Growth curve measurements show that each cranberry proanthocyanidin fraction (without antibiotic) did not reduce the growth rates of E. coli CFT073 and P. aeruginosa PAO1 when compared to untreated cells of each strain (FIG. 3). This demonstrates that the observed bioactivity of the cranberry proanthocyanidins extract is not a killing effect but rather a synergism with the antibiotic.

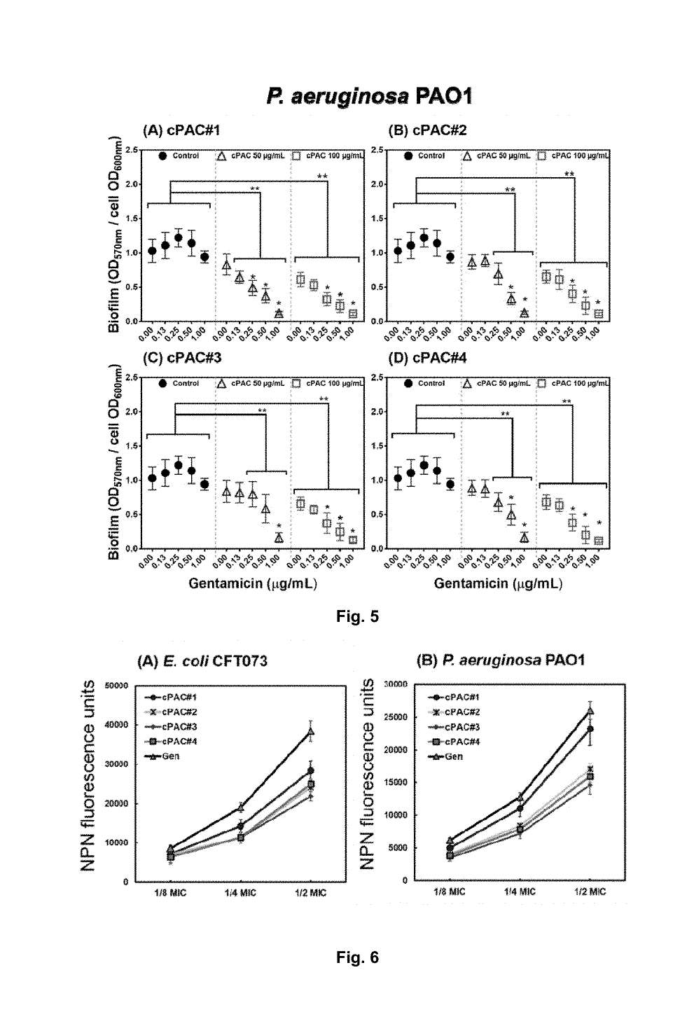

Cranberry proanthocyanidins also significantly reduced biofilm formation formed by each pathogenic bacterial strain at sub-lethal concentrations (see FIGS. 4 and 5). Proanthocyanidins derived from cranberry cause cell membrane permeabilization and efflux pump inhibition of pathogenic bacteria without affecting cell membrane integrity.

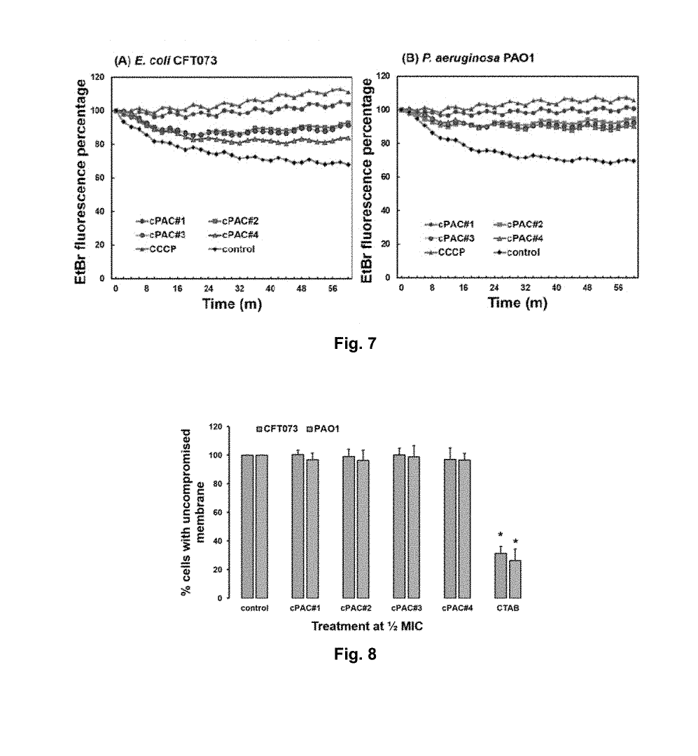

The specific mechanism(s) of action for the observed synergistic interactions between proanthocyanidins and antibiotic is disclosed. As mentioned hereinabove, the proanthocyanidins at sub-inhibitory concentrations permeabilize the cell outer-membrane and inhibit multidrug resistance efflux pumps involved in multidrug resistance in pathogenic bacteria, without affecting cell membrane integrity (see FIGS. 6-8). This is interesting, because elimination of persister cells at sub-inhibitory concentrations of cranberry proanthocyadins can reduce the amount of antibiotic required for the treatment of chronic and recurrent infections. The beneficial properties of cranberry proanthocyanidins suggest that the combination of the natural compounds and antibiotics may be an effective new anti-bacterial therapy.

Encompassed herein is the combination of the cranberry extract and composition described herein with an antibiotic. For example, but not limited to, the antibiotic can be an aminoglycoside, a polyketide, a macrolide, a fluoroquinolone or a β-lactam, more specifically, the antibiotic can be gentamicin, kanamycin, tetracycline, or azithromycin.

Also encompassed is the combination of the cranberry extract and composition described herein with different materials used in the art for non-limiting application in medical settings such as natural anti-infective, anti-microbial, anti-biofilm or anti-virulence agent in individual or combinatorial therapies thereof.

Further encompassed is the combination of the cranberry extract and composition described herein with materials use for non-limiting applications such as edible or non-edible functional or non-functional food coatings or food packaging

The present disclosure will be more readily understood by referring to the following examples.

### Example I

### Minimum Inhibitory Concentration (MIC)

Two organisms were used to demonstrate the efficacy of the composition described herein: E. coli strain CFT073 (ATCC 700928) and P. aeruginosa PAO1 (ATCC 15692). Pure stock cultures were maintained at −80° C. in 30% (v/v) frozen glycerol solution. Starter cultures were prepared by streaking frozen cultures onto LB agar (LB broth: tryptone 10 g/L, yeast extract 5 g/L and NaCl 5 g/L, supplemented with 1.5 w/v % agar (Fisher Scientific, Canada)). After overnight incubation at 37° C., a single colony was inoculated into 10 mL of LB broth and the culture was incubated at 37° C. on an orbital shaker at 150 rpm for time lengths specific to each experiment. LB broth was used for bacterial culture in all experiments unless otherwise specified.

Minimum Inhibitory Concentration (MIC) was determined by preparing two-fold serial dilutions of each cPACs fraction and antibiotic in Mueller Hinton broth adjusted with Ca²⁺ and Mg²⁺ (MHB-II, Oxoid, Fisher Scientific, Canada). A range of concentration of the antibiotics gentamicin (0.0156-2 μg/mL), tetracycline (0.03-4 μg/mL), kanamycin (0.25-512 μg/mL), azithromycin (0.125-256 μg/mL), ciprofloxacin (0.0003-1 μg/mL) and ampicillin (0.25-2000 μg/mL), was used. Dilutions were prepared in flat bottom, 96 well microtitre plates (Falcon, Corning, Fisher Scientific, Canada). Each well of a microtitre plate was then inoculated with the desired bacterial strain (grown in MHB-II and diluted to 10⁶ CFU/mL) and the plate was incubated at 37° C. for 18 hours under static conditions. Bacterial growth was assessed by (i) monitoring the optical density of the cell suspension in each well at 600 nm (OD600 nm), and (ii) the resazurin microtitre plate assay. In the resazurin microtitre plate assay, each well of a microtitre plate was supplemented with 20 μM resazurin, incubated in dark for 20 min at room temperature, followed by fluorescence measurements at ex/em 570/590 nm using a TECAN Infinite M200 Pro microplate reader (Tecan Group Ltd., Switzerland). The lowest concentration of a compound able to prevent increase in OD600 nm and resazurin fluorescence intensity was recorded as the MIC for that compound.

### Example II

### Checkerboard Microdilution Assay

The checkerboard microdilution assay was used for evaluation of in vitro antimicrobial synergy between two compounds (i.e., antibiotic and each cPAC fraction). Two-fold serial dilutions were prepared in MHB-II for each of the two compounds under study. The serial dilutions were then loaded into 96 well plates to achieve combinations having different concentrations of each of the two compounds. Each well was subsequently inoculated with 10⁶ CFU/mL of the desired bacterial strain and incubated at 37° C. for 18 hours under static conditions. The Fractional Inhibitory Concentration Index (FICI) for each combination was calculated by using the following formulae:

FIC_{component 1}=MIC_{component1,in combination}/MIC_{component1,alone}

FICI=FIC_{component 1}+FIC_{component 2}

The FICIs were interpreted as follows: FICI of ≤0.5 (synergy); 0.5<FICI≤4 (no interaction/indifference); FICI of >4 (antagonism).

### Example III

### Biofilm Formation

Biofilm formation was quantified using the standard microtitre plate model. Briefly, overnight cultures (MHB-II broth, 37° C., 200 rpm) were diluted 1:100 (v/v) into fresh MHB-II broth (with or without each cPAC fraction and their combination with gentamicin), to 10⁶ CFU/mL. Aliquots (100 μL) of these cultures were transferred into the wells of polystyrene, flat bottom, non-treated 96 well plates (Falcon, Corning), in triplicate. For all assays, biofilms were allowed to develop for 18 hours at 37° C. under static conditions, after which OD600 values were recorded, the spent broth was decanted from the wells and the wells were gently rinsed three times with DI water. The washed biofilm was stained with crystal violet (CV). For CV stain assay, 100 μL of 0.1% (w/v) CV was loaded in each well and the plates were incubated for 15 minutes under static condition at room temperature. The wells were subsequently rinsed with DI water to remove excess dye and the CV adsorbed to the biomass in each well was solubilized in 100 μL of absolute ethanol for 10 minutes. The solubilized CV was then quantified (as OD570) using a microplate reader. Control experiments were performed with cell-free broth to adjust for background signal.

### Example IV

### Membrane Permeabilization and Membrane Integrity Assays

The outer membrane permeabilization activities of each cPAC fraction and antibiotic were determined by the 1-N-phenylnapthylamine (NPN, Sigma-Aldrich Canada) assay with some modifications. Briefly, overnight bacterial cultures were diluted 1:1 in MHB-II medium to a final volume of 10 mL, with or without sub-MIC supplementation of each cPACs fraction or gentamycin (as a positive control), and grown to an OD600 of 0.5-0.6 (37° C., 200 rpm). The cells were harvested, washed with 5 mM HEPES buffer (pH 7.2), and resuspended in the same volume (10 mL) of 5 mM HEPES buffer (pH 7.2) containing 1 mM N-ethylmaleimide (NEM, Sigma-Aldrich Canada). Aliquots (1 mL) were mixed with NPN to a final concentration of 10 μM (in cell suspension) and fluorescence was measured using the microplate reader (ex/em 350/420 nm).

The BacLight kit (L-13152, Invitrogen, Life Technologies Inc., Canada) was used to assess cell membrane damage. Overnight bacterial cultures were diluted 1:40 in fresh MHB-II broth to a final volume of 5 mL, grown to an OD600 of 0.5-0.6, washed with filter-sterilized 10 mM phosphate buffered saline (PBS, pH 7.0) and resuspended in 1/10 of the original volume. The washed cells were then diluted 1:20 v/v into stock solution of each cPACs fraction at ½ MICs or DI water (control). Cultures were incubated at room temperature (27±2° C.) on a tube rocker for 10 minutes. At the end of the incubation period, an aliquot was taken for CFU counts and the remaining suspension was washed with 10 mM PBS and resuspended to an OD600 of 0.3. An aliquot (100 μL) of each bacterial suspension was removed and added to a 96-well, black, clear-bottom plate (Corning, Fisher Scientific, ON, Canada) along with an equal volume of the BacLight reagent (2× stock solution, L13152, Invitrogen, Life Technologies Inc., Canada) and the plates were incubated for 10 minutes at room temperature in the dark. At the end of the incubation period, fluorescence intensity was recorded for both kit components, SYTO-9 (ex/em 485/530 nm) and propidium iodide (ex/em 485/645 nm), using the microplate reader. Fluorescence readings from samples were normalized to the values obtained from untreated control to determine the ratio of membrane compromised cells to cells with intact membrane. CTAB (Sigma-Aldrich Canada), a cationic detergent that is known to cause membrane damage, was used at concentration of 10 μM as a positive control for membrane disruption.

### Example V

### Ethidium Bromide (EtBr) Efflux Assay

To assess the effect of each cPAC fraction on the inhibition of the proton motive force driven multidrug efflux pump, an ethidium bromide (EtBr) efflux assay was performed. An overnight grown culture of each strain was diluted 1:100 using MHB-II broth to a final volume of 10 mL and grown to an OD600 of 0.8-1.0 (at 37° C., 150 rpm). Cells were loaded in polystyrene microcentrifuge tubes (2 mL) and mixed with 5 μM EtBr and each cPAC fraction at 25% of their MIC, or 100 μM of the proton conductor, carbonyl cyanide m-chlorophenylhydrazone (CCCP, Sigma-Aldrich Canada), as positive control. Replica tubes that did not receive cPAC or proton conductor served as negative controls. The tubes were incubated for 1 hour (37° C., 150 rpm). The inoculum was then adjusted to 0.4 OD600 with MHB-II broth containing 5 μM EtBr and 2 mL aliquots of this mixture were pelleted (5000×g, 10 min at 4° C.). The pellets were incubated on ice immediately, resuspended in 1 mL of MHB-II and aliquoted (200 μL) into a polystyrene 96 well, black, clear-bottom plate (Corning, Fisher Scientific, Canada). EtBr efflux from the cells was monitored at room temperature using the microplate reader (ex/em 530/600 nm). Readings were taken at 5 minute intervals for 1 hour to monitor efflux pump activity. The background fluorescence of the medium was subtracted from all measurements and the assay was repeated independently in triplicate.

While the invention has been described in connection with specific embodiments thereof, it will be understood that it is capable of further modifications and this application is intended to cover any variations, uses, or adaptations of the invention, including such departures from the present disclosure as come within known or customary practice within the art to which the invention pertains and as may be applied to the essential features hereinbefore set forth, and as follows in the scope of the appended claims.

1. A synergistically active composition comprising a cranberry extract and at least one antibiotic for treating a bacterial infection.

2. The composition of claim 1, wherein the cranberry extract comprises proanthocyanidins, flavanols, anthocyanidins, procyanidins, terpenes, hydroxybenzoic acids, hydroxycinnamic acids, flavonoids, tannins, phenolic acids, other bioactive molecules or combinations thereof.

3. The composition of claim 1, wherein the cranberry extract is from at least one of Vaccinium macrocarpon, Vaccinium oxycoccos, Vaccinium microcarpum, and Vaccinium erythrocarpum.

4. The composition of claim 1, wherein the cranberry extract is from Vaccinium macrocarpon.

5. The composition of claim 1, wherein the at least one antibiotic is an aminoglycoside, a polyketide, a macrolide, a fluoroquinolone, a benzenoid, an azolidine, an organic phosphonic acid, a β-lactam or their derivatives and combinations thereof.

6. The composition of claim 1, wherein the at least one antibiotic is gentamicin, kanamycin, tetracycline, azithromycin, trimethoprim, sulfamethoxazole, nitrofurantoin, norfloxacin, fosfomycin, ciprofloxacin or their combinations thereof.

7. The composition of claim 1, wherein the at least one antibiotic is trimethoprim and sulfamethoxazole.

8. The composition of claim 1, comprising 95% proanthocyanidins.

9. The composition of claim 1, wherein the bacterial infection is from E. coli, P. mirabilis, P. aeruginosa, Burkholderia ambifaria, Chromobacterium violaceum or Enterococcus faecalis.

10. The composition of claim 1, wherein said composition is a quorum sensing (QS) inhibitor.

11. The composition of claim 1, wherein said composition permeabilizes cell membranes to the at least one antibiotic.

12. The composition of claim 1, wherein said composition inhibits efflux pumps.

13. The composition of claim 1 wherein said composition enhances membrane transport of tetracycline.

14. The composition of claim 1, wherein said composition is an antagonist of LasR or RhIR.

15. The composition of claim 1, for treating a urinary tract infection.

16-32. (canceled)

33. A method of treating a bacterial infection, comprising administering the synergistically active composition of claim 1 to a subject.

34-42. (canceled)

43. The method of claim 33, wherein said composition permeabilizes cell membranes to the at least one antibiotic.

44. The method of claim 33, wherein said composition inhibits efflux pumps.

45. The method of claim 33, wherein said composition enhances membrane transport of tetracycline.

46. (canceled)

47. The method of claim 33, for treating a urinary tract infection.
